(19) United States
(12) Patent Application Publication
(10) Pub. No.: US 2020/0188023 A1
(43) Pub. Date: Jun. 18, 2020

(54) INFORMATION PROCESSING APPARATUS, INFORMATION PROCESSING METHOD, AND PROGRAM

(71) Applicant: SONY CORPORATION, TOKYO (JP)

(72) Inventor: YOSHIO SOMA, KANAGAWA (JP)

(21) Appl. No.: 16/603,029

(22) PCT Filed: Mar. 16, 2018
(86) PCT No.: PCT/JP2018/010474

§ 371 Date: Oct. 4, 2019

(30) Foreign Application Priority Data
Apr. 21, 2017 (JP) 2017-084674

(51) Int. Cl. A61B 34/10; A61F 9/007; A61B 3/10; G16H 20/40; G16H 30/40; G02B 21/00; G02B 21/36

(52) U.S. Cl. CPC A61B 34/10; A61F 9/007; A61B 3/102; A61F 9/00754; A61B 90/10; G16H 30/40; G02B 21/0012; G02B 21/365; A61B 2034/107; G16H 20/40

(57) ABSTRACT

[Object] To provide a technology by which a suitable parameter for acquiring a tomographic image that meets a user's demand can be set. [Solving Means] An information processing apparatus according to the present technology includes a control unit. The control unit detects a time-series change of an object shown in an image obtained by taking an image of an eye which is a surgical operation target and changes a parameter for acquiring a tomographic image of the eye in accordance with the time-series change of the object.

### TECHNICAL FIELD

The present technology relates to technologies such as an information processing apparatus that executes processing related to a tomographic image of an eye which is displayed during surgical operation for the eye.

### BACKGROUND ART

In recent years, surgical microscope apparatuses have been widely used in the surgical operation on the eye. This surgical microscope apparatus causes an image of the eye which is acquired via a microscope and a tomographic image of the eye which is acquired by an optical coherence tomography (OCT) or the like to be displayed. A user performs a surgical operation for the eye while referring to those images. With this configuration, generation of surgical errors is prevented and, in addition, surgical operation accuracy is improved.

The OCT is a technology of irradiating an eye with a near-infrared ray and reconstructing light reflected by respective tissues of the eye to generate an image. With the OCT, a tomographic image of the eye in a particular tomographic surface can be obtained. For example, Patent Literature 1 below has disclosed an ophthalmologic analysis apparatus that presents a tomographic image of an eye, which is acquired by the OCT, to a user.

### CITATION LIST

### Patent Literature

Patent Literature 1: Japanese Patent Application Laid-open No. 2014-140490

### DISCLOSURE OF INVENTION

### Technical Problem

In acquisition of a tomographic image by the OCT or the like, a user needs to adjust a number of parameters in order to obtain a tomographic image that meets a demand of that user. However, work of setting those parameters to take suitable values is work which is time-consuming and requires experience for the user, and that work is a large burden on the user.

In view of the above-mentioned circumstances, it is an object of the present technology to provide a technology by which a suitable parameter for acquiring a tomographic image that meets a user's demand can be set.

### Solution to Problem

In order to accomplish the above-mentioned object, the information processing apparatus according to the present technology includes a control unit. The control unit detects a time-series change of an object shown in an image obtained by taking an image of an eye which is a surgical operation target and changes a parameter for acquiring a tomographic image of the eye in accordance with the time-series change of the object.

In this manner, by changing the parameter for acquiring the tomographic image of the eye in accordance with the time-series change of the object, it is possible to set a suitable parameter for acquiring a tomographic image that meets a user's demand.

In the information processing apparatus, the control unit may detect a change in position of a surgical tool as the time-series change of the object and changes the parameter in accordance with the change in position of the surgical tool.

With this configuration, it is possible to set a suitable parameter for acquiring a tomographic image that meets a user's demand.

In the information processing apparatus, the control unit may detect a change in condition of the eye as the time-series change of the object and changes the parameter in accordance with the change in condition of the eye.

With this configuration, it is possible to set a suitable parameter for acquiring a tomographic image that meets a user's demand.

In the information processing apparatus, the control unit may switch between a first mode on which the parameter is set such that priority is given to a frame rate over an image quality in the tomographic image and a second mode on which the parameter is set such that priority is given to the image quality over the frame rate in the tomographic image in accordance with the time-series change of the object.

With this configuration, two modes can be suitably switched so as to meet a user's demand.

In the information processing apparatus, the control unit may detect a change speed in the time-series change of the object and switches between the first mode and the second mode in accordance with the change speed.

With this configuration, two modes can be suitably switched so as to meet a user's demand.

In the information processing apparatus, the control unit may determine whether or not the change speed is equal to or higher than a predetermined threshold, may set the first mode if the change speed is equal to or higher than the predetermined threshold, and may set the second mode if the change speed is lower than the predetermined threshold.

With this configuration, two modes can be suitably switched so as to meet a user's demand.

In the information processing apparatus, the control unit may set, in accordance with the time-series change of the object, a first region within a measurement region in which scan for obtaining the tomographic image is performed and change the parameter between the first region and a second region, the second region being a region other than the first region within the measurement region.

With this configuration, the parameter for acquiring the tomographic image can be suitably changed between the first region and the second region so as to meet a user's demand.

In the information processing apparatus, the control unit may change scan density in the scan as the parameter between the first region and the second region.

With this configuration, the parameter (scan density) for acquiring the tomographic image can be suitably changed between the first region and the second region so as to meet a user's demand.

In the information processing apparatus, the control unit may change the scan density in the scan such that scan density of the first region is higher than scan density of the second region.

With this configuration, the parameter (scan density) for acquiring the tomographic image can be suitably changed between the first region and the second region so as to meet a user's demand.

In the information processing apparatus, the control unit may change a frame rate in the tomographic image as the parameter between the first region and the second region.

With this configuration, the parameter (frame rate) for acquiring the tomographic image can be suitably changed between the first region and the second region so as to meet a user's demand.

In the information processing apparatus, the control unit may change a frame rate in the tomographic image such that a frame rate in the first region is higher than a frame rate in the second region.

With this configuration, the parameter (frame rate) for acquiring the tomographic image can be suitably changed between the first region and the second region so as to meet a user's demand.

In the information processing apparatus, the control unit may change the tomographic surface for acquiring the tomographic image as the parameter on the basis of the time-series change of the object.

With this configuration, the tomographic surface can be set at a suitable position.

In the information processing apparatus, the control unit may predict a change of the object at a time later than a current time by a predetermined time in accordance with the time-series change of the object and change the tomographic surface in accordance with a prediction result.

With this configuration, the tomographic surface can be set at a suitable position.

An information processing method according to the present technology includes: detecting a time-series change of an object shown in an image obtained by taking an image of an eye which is a surgical operation target; and changing a parameter for acquiring a tomographic image of the eye in accordance with the time-series change of the object.

A program according to the present technology causes a computer to execute: a step of detecting a time-series change of an object shown in an image obtained by taking an image of an eye which is a surgical operation target; and a step of changing a parameter for acquiring a tomographic image of the eye in accordance with the time-series change of the object.

### Advantageous Effects of Invention

As described above, in accordance with the present technology, it is possible to provide a technology by which a suitable parameter for acquiring a tomographic image that meets a user's demand can be set.

### BRIEF DESCRIPTION OF DRAWINGS

FIG. 1 A block diagram showing a configuration of a surgical microscope apparatus according to a first embodiment.

FIG. 2 A schematic diagram showing a cataract surgery process.

FIG. 3 A schematic diagram showing a cataract surgery process.

FIG. 4 A flowchart showing processing of a control unit.

FIG. 5 A diagram showing an example of a front image acquired by a front image acquisition unit.

FIG. 6 A diagram showing an example of a tomographic image acquired by a tomographic image acquisition unit.

FIG. 7 A flowchart showing processing according to a second embodiment.

FIG. 8 A diagram showing an example of a track of a motion of a distal end of a surgical tool from a time earlier than a current time by a predetermined time to the current time.

FIG. 9 A diagram showing a state when a region of interest is set on the basis of a motion of the distal end of the surgical tool.

FIG. 10 A diagram showing a state when a region of interest is set on the basis of a motion of the distal end of the surgical tool.

FIG. 11 A diagram showing a state when a region of interest is set within a measurement region.

FIG. 12 A diagram showing a scan pattern for acquiring the tomographic image.

FIG. 13 A flowchart showing processing in a case of making a frame rate of a region of interest and a frame rate of a region of non-interest different.

FIG. 14 A diagram showing a state when the region of interest is set on the basis of a change in condition of an eye.

FIG. 15 A diagram showing a state when a region of interest is set on the basis of a change in condition of the eye.

FIG. 16 A flowchart showing processing according to a third embodiment.

FIG. 17 A diagram showing a state when a position of the distal end of the surgical tool at a time later than the current time by a predetermined time is predicted.

FIG. 18 A diagram showing an example of a tomographic surface determined on the basis of a prediction result.

FIG. 19 A diagram showing an example of a tomographic surface determined on the basis of a prediction result.

### MODE(S) FOR CARRYING OUT THE INVENTION

Hereinafter, embodiments according to the present technology will be described with reference to the drawings.

### First Embodiment

### <Overall Configuration and Configuration of Respective Units>

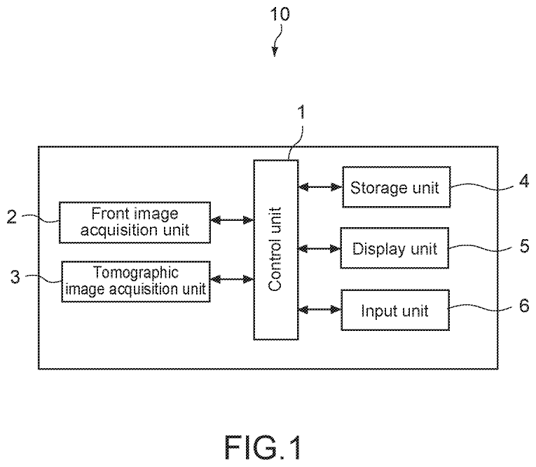

FIG. 1 is a block diagram showing a configuration of a surgical microscope apparatus 10 according to a first embodiment. As shown in FIG. 1, the surgical microscope apparatus 10 (information processing apparatus) includes a control unit 1, a front image acquisition unit 2, a tomographic image acquisition unit 3, a storage unit 4, a display unit 5, and an input unit 6.

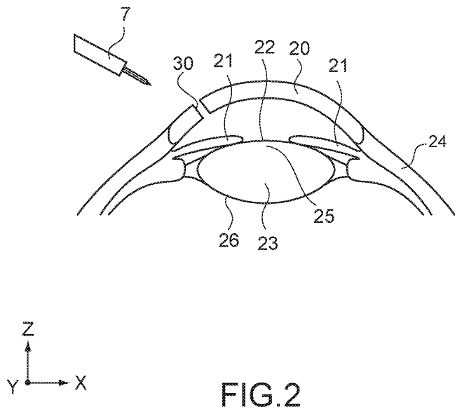

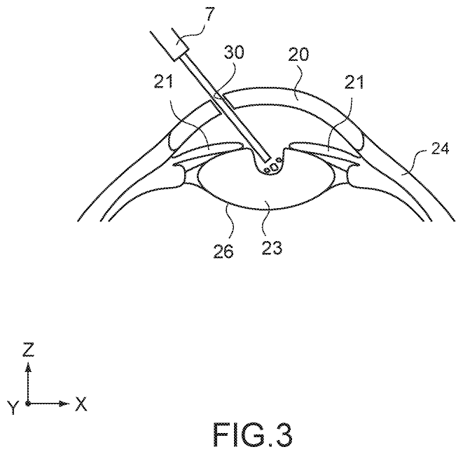

It should be noted that in the description of this embodiment, it is assumed that a depth direction of an eye is a Z-axis direction and arbitrary directions in a plane direction of the eye are an X-axis direction and a Y-axis direction (see FIGS. 2 and 3, etc. to be described later).

The front image acquisition unit 2 includes, for example, a microscope apparatus with a camera, a microscope apparatus with a stereo camera, and the like. The front image acquisition unit 2 takes an image of an eye which is a surgical operation target from the front, acquires a front image, and outputs the acquired front image to the control unit 1.

The tomographic image acquisition unit 3 includes an OCT in this embodiment. It should be noted that the tomographic image acquisition unit 3 may include a Scheimpflug camera. The tomographic image acquisition unit 3 irradiates the eye with a near-infrared ray and utilizes light interference of reflected light from the eye and reference light to execute scan in a depth direction (Z-axis direction) of the eye.

The tomographic image is configured in such a manner that rectangular images very elongated in the depth direction (hereinafter, rectangular images) are arranged along an arbitrary direction in a plane direction (XY direction). Therefore, the tomographic image acquisition unit 3 is configured to be capable of not only scanning in the depth direction but also scanning in any one direction in at least the plane direction.

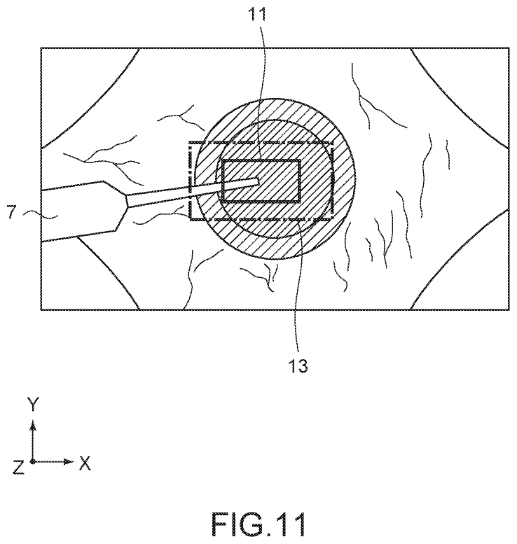

The tomographic image acquisition unit 3 acquires a tomographic image on the basis of various parameters. Here, the various parameters include a measurement position, a measurement region 13 (see FIG. 11 to be described later), scan density, the average number of processing times, a tomographic surface, and the like.

The measurement position is a position in which the measurement by the OCT is performed. The measurement region 13 is a region in the plane direction in which the measurement by the OCT is performed (a region in the plane direction in which scan is performed).

Here, the measurement region 13 may be set to be linear (scan line) as the eye is viewed from above (Z-axis direction) or may be set to be planar as the eye is viewed from above. In a case where the measurement region 13 is set to be planar, the volume data (three-dimensional data) of the eye can be obtained.

The measurement region 13 is fixed in this embodiment. It should be noted that the measurement region 13 may be set in accordance with a user's instruction. The scan density is density of scan in the plane direction (a value indicating at which intervals the measurement by the OCT is performed in the plane direction).

The average number of processing times is the number of continuous image acquisition times in processing of removing random noise by continuously acquiring images at the same position and averaging respective pixel values. Further, the tomographic surface is a surface which is a criteria when generating a tomographic image. That is, the tomographic image is generated in such a manner that the rectangular images are arranged along a direction on the tomographic surface by using the tomographic surface as a reference. The tomographic surface may be specified by the user or may be determined on the basis of a position of a surgical tool 7 (see FIGS. 2 and 3).

It should be noted that in a case where the measurement region 13 is set to be linear (scan line), the direction of the tomographic surface is identical to a linear measurement region 13. In this case, only by simply arranging the acquired rectangular images, the tomographic image is generated. On the other hand, in a case where the measurement region 13 is set to be planar, rectangular images of rectangular images acquired in the planar measurement region 13, which are located in a direction along the tomographic surface, are picked up and the picked up rectangular images are arranged. The tomographic image is thus generated.

The display unit 5 includes a liquid-crystal display, an organic electro luminescence (EL) display, and the like. The display unit 5 causes a front image of the eye acquired by the front image acquisition unit 2 and a tomographic image of the eye acquired by the tomographic image acquisition unit 3 to be displayed on the screen.

The input unit 6 includes, for example, a keyboard, a mouse, a touch sensor provided on the screen of the display unit 5, and the like. The input unit 6 inputs a user's operation signal and outputs the input user's operation signal to the control unit 1.

The control unit 1 includes a central processing unit (CPU) and the like. The control unit 1 executes various types of calculation based on various programs stored in the storage unit 42 and comprehensively controls the respective units of the surgical microscope apparatus 10. It should be noted that processing of the control unit 1 will be described later in detail in the section of the operation description.

The storage unit 42 includes a nonvolatile memory in which various programs and various types of data required for the processing of the control unit 1 are stored and a volatile memory to be used as a working area for the control unit 1. It should be noted that various programs stored in the storage unit 42 may be read from a portable recording medium such as an optical disc and a semiconductor memory or may be downloaded from a server apparatus over a network.

<Outline of Ophthalmologic Surgery>

Next, the outline of the cataract surgery in which the surgical microscope apparatus 10 can be utilized will be described. FIGS. 2 and 3 are schematic diagrams each showing a cataract surgery process. As shown in those figures, the eyeball includes tissues such as a cornea 20, an iris 21, a lens 23, and a sclera 24. An anterior capsule 25 is formed on a front surface in the lens 23. A posterior capsule 26 is formed on a rear surface in the lens 23. At the back of the lens 23, a vitreous body (not shown) is located. Further, it is a pupil 22 that is located in the middle of the iris 21 on the surface of the lens 23.

As shown in FIG. 2, in the cataract surgery, a surgical wound 30 is formed in the cornea 20 through a surgical tool 7 such as a scalpel. Next, the surgical tool 7 is inserted through the surgical wound 30 and the anterior capsule 25 in the lens 23 is incised through the surgical tool 7, an interior (core or cortex) of the lens 23 is absorbed and removed through a surgical tool 7 for absorption as shown in FIG. 3. After that, an intraocular lens is inserted at the position at which the lens 23 has been removed. The surgery is thus completed.

It should be noted that the cataract surgery shown here is an example of ophthalmologic surgery in which the surgical microscope apparatus 10 can be utilized and the surgical microscope apparatus 10 can be utilized in various types of ophthalmologic surgery.

<Operation Description>

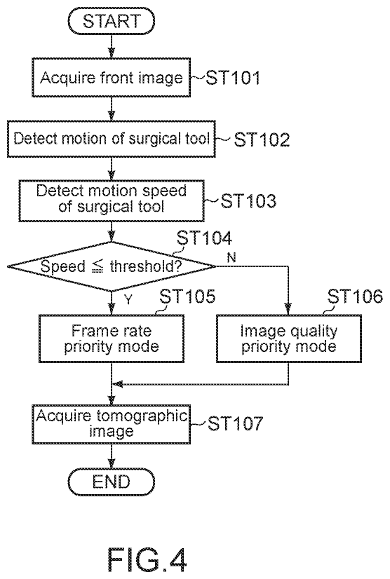

Next, processing of the control unit according to this embodiment will be described. FIG. 4 is a flowchart showing the processing of the control unit 1. As shown in FIG. 4, first of all, the control unit 1 controls the front image acquisition unit 2 to capture a front image of an eye and acquires the captured front image of the eye from the front image acquisition unit 2 (Step 101).

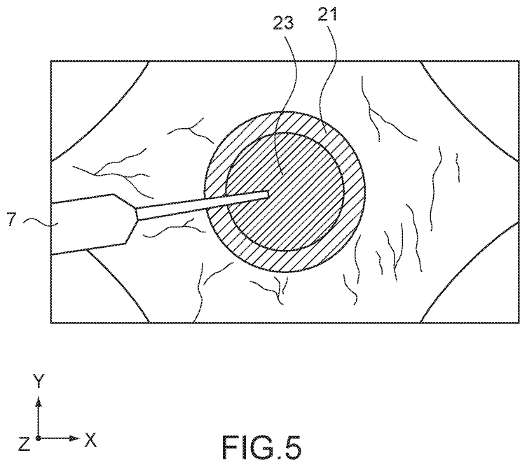

FIG. 5 is a diagram showing an example of the front image acquired by the front image acquisition unit 2. In the front image shown in FIG. 5, a state when the interior of the lens 23 is absorbed and removed through a surgical tool 7 for absorption in the cataract surgery is shown.

It should be noted that every time the front image is acquired, the control unit 1 causes the storage unit 4 to store the acquired front image. Further, the control unit 1 causes the acquired front image to be displayed on the screen of the display unit 5.

Next, the control unit 1 executes image recognition processing on the previous front image stored in the storage unit 4 and the acquired current front image and detects a motion of the surgical tool 7 shown in the front image (time-series change of the object: change in position of the object) (Step 102).

Examples of a method to be used for image recognition processing can include an inter-frame difference method, template matching, a feature point extracting/tracking method, and the like. It should be noted that the motion of the surgical tool 7 may be detected on the basis of an optical or magnetic maker provided in a distal end portion, gripping portion, or the like of the surgical tool 7. Further, the motion of the surgical tool 7, which is to be detected, may be a motion of the entire surgical tool 7 or may be a motion of the distal end of the surgical tool 7.

Next, the control unit 1 detects a speed of the motion of the surgical tool 7 (speed of the change in position) on the basis of the detected motion of the surgical tool 7 (Step 103). The speed of the motion of the surgical tool 7 may be a speed of the motion of the entire surgical tool 7 or may be a speed of the motion of the distal end of the surgical tool 7.

Next, the control unit 1 determines whether or not the speed of the motion of the surgical tool 7 is equal to or higher than a predetermined threshold (Step 104). If the speed of the motion of the surgical tool 7 is equal to or higher than the predetermined threshold (YES in Step 104), the control unit 1 sets the mode in the tomographic image to be a frame rate priority mode (first mode) (Step 105). The frame rate priority mode is a mode on which priority is given to the frame rate (refresh rate: the number of updating times of the tomographic image per unit time) over the image quality in the tomographic image.

On the frame rate priority mode, the control unit 1 sets the scan density to be a lower value, reduces the average number of processing times, and makes the frame rate higher in the parameter in the tomographic image, for example. On the frame rate priority mode, the image quality is becomes lower (lower resolution) while the frame rate can be made higher. It should be noted that various parameters on the frame rate priority mode are set in advance and a tomographic image is acquired using these various parameters.

Here, on the basis of the fact that the motion of the surgical tool 7 is speedy, it can be considered that a change in condition of the eye which is the surgical operation target is also speedy. In such a case, it can be considered that the demand of quickly grasping a change in condition of the eye from the tomographic image highly frequently updated is stronger than the demand of specifically observing the condition of the eye from the high-quality tomographic image from the perspective of the user. Therefore, in this embodiment, when the motion of the surgical tool 7 is speedy, the frame rate priority mode on which priority is given to the frame rate over the image quality is adapted to be set.

On the other hand, in Step 104, if the speed of the motion of the surgical tool 7 is lower than the predetermined threshold (NO in Step 104), the control unit 1 sets the mode in the tomographic image to be the image quality priority mode (second mode) (Step 106). The image quality priority mode is a mode on which priority is given to the image quality over the frame rate in the tomographic image.

On the image quality priority mode, the control unit 1 sets the scan density to be a higher value, increases the average number of processing times, and makes the image quality higher in the parameter in the tomographic image, for example. On the image quality priority mode, the frame rate is lower while the image quality can be made higher (higher resolution). It should be noted that various parameters on the image quality priority mode are set in advance and a tomographic image is acquired using these various parameters.

It should be noted that the surgical tool 7 is not necessarily constantly shown in the front image. If the control unit 1 determines that the surgical tool 7 is not shown in the front image, the control unit 1 sets the mode in the tomographic image to be the image quality priority mode in a manner similar to that performed if the speed of the motion of the surgical tool 7 is lower than the predetermined threshold.

Here, on the basis of the fact that the motion of the surgical tool 7 is slow, it can be considered that a change in condition of the eye which is the surgical operation target is also slow. Further, on the basis of the fact that the surgical tool 7 is not shown in the front image, it can be considered that it may be before start of the surgical operation, the surgical operation may be temporarily stopped during surgical operation, and it may be after the surgical operation. Also in this case, it can be considered that a change in condition of the eye which is the surgical operation target is also slow.

In such a case, it can be considered that from the perspective of the user, the demand of specifically observing the condition of the eye from the high-quality tomographic image (before surgical operation, during surgical operation, or after surgical operation) is stronger than the demand of quickly grasping a change in condition of the eye from the tomographic image highly frequently updated. Therefore, in this embodiment, when the motion of the surgical tool 7 is slow (or when the surgical tool 7 is not shown in the front image), the image quality priority mode on which priority is given to the image quality over the frame rate is adapted to be set.

When the control unit 1 sets the mode, the control unit 1 causes the tomographic image acquisition unit 3 to acquire a tomographic image on the basis of the parameter according to the set mode (Step 107). When the tomographic image is acquired, the control unit 1 causes the acquired tomographic image to be displayed on the screen of the display unit 5.

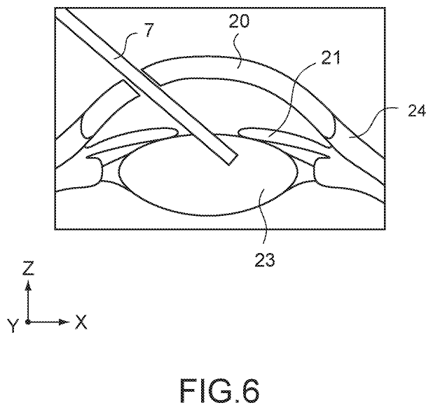

FIG. 6 is a diagram showing an example of the tomographic image acquired by the tomographic image acquisition unit 3. In the tomographic image shown in FIG. 6, a state when the interior of the lens 23 is absorbed and removed by the surgical tool 7 for absorption in the cataract surgery is shown. It should be noted that the front image as shown in FIG. 5 and the tomographic image as shown in FIG. 6 may be displayed on the same display unit 5 or may be displayed on discrete display units 5.

<Actions, Etc.>

Here, a case where the user sets the parameter in the tomographic image by that user self and controls the image quality and the frame rate will be assumed as a comparison. As the parameter in the tomographic image, there are various parameters as described above and these parameters are complicated associated with the image quality and the frame rate. Therefore, the work of setting the various parameters to take suitable values in order to obtain both of the image quality and the frame rate that meet a user's demand is work which is time-consuming and requires experience for the user, and that work is a large burden on the user.

In addition, during surgical operation, in accordance with progress of the surgical operation, the condition of the eye changes or the user's demand on the tomographic image changes. Therefore, it is necessary to more frequently adjust parameters. The frequent adjustment of the parameters during surgical operation causes frequent intervention in the surgical operation. Further, the frequent adjustment of the parameters causes lowering of powers of concentration or work efficiency of the user and leads to delay of the surgical operation time. Further, the effect obtained by displaying the tomographic image cannot be sufficiently exerted if suitable parameters cannot be set.

On the other hand, in this embodiment, the parameter for acquiring the tomographic image is changed on the basis of the motion of the surgical tool 7 in the front image (i.e., the change in position of the object). That is, in this embodiment, the correlation between the motion (speed) of the surgical tool 7 and the user's demand on the tomographic image is considered and the parameters of the tomographic image are changed in accordance with the motion of the surgical tool 7 so as to meet a user's demand.

Therefore, in this embodiment, in accordance with the motion of the surgical tool 7, it is possible to set a suitable parameter for acquiring a tomographic image that meets a user's demand. In particular, in this embodiment, even when the user's demand in the tomographic image changes in progress condition in the surgical operation (e.g., before surgical operation, during surgical operation, after surgical operation, or the like), the parameters can be suitably changed (because the relationship between the motion of the surgical tool 7 and the demand is utilized) in accordance with this demand.

Further, in this embodiment, the control unit 1 automatically changes the parameter for acquiring the tomographic image. Therefore, the burden in changing the parameters by the user can be reduced. In particular, in this embodiment, the parameters are automatically changed also during surgical operation. Therefore, it is possible to prevent intervention in the surgical operation, lowering of powers of concentration or work efficiency of the user, and leading to delay of the surgical operation time during surgical operation.

Further, in this embodiment, the frame rate priority mode is set if the speed of the motion of the surgical tool 7 is equal to or higher than the predetermined threshold and the image quality priority mode is set if the speed of the motion of the surgical tool 7 is lower than the predetermined threshold. With this configuration, in this embodiment, two modes can be suitably switched so as to meet a user's demand.

### Modified Example of First Embodiment

In the description above, the case where the parameter for acquiring the tomographic image is changed has been described in accordance with the motion of the surgical tool 7. On the other hand, the parameter for acquiring the tomographic image may be changed in accordance with the change in condition of the eye shown in the front image.

As described above, when the change in condition of the eye which is the surgical operation target is speedy, it can be considered that the demand of quickly grasping a change in condition of the eye from the tomographic image highly frequently updated is stronger than the demand of specifically observing the condition of the eye from the high-quality tomographic image from the perspective of the user.

Further, as described above, when the change in condition of the eye which is the surgical operation target is slow, it can be considered that from the perspective of the user, the demand of specifically observing the condition of the eye from the high-quality tomographic image (before surgical operation, during surgical operation, or after surgical operation) is stronger than the demand of quickly grasping a change in condition of the eye from the tomographic image highly frequently updated.

That is, there is also a correlation between the change in condition of the eye and the user's demand and this relationship is utilized. Examples of the change in condition of the eye can include a change when the surgical wound 30 is formed in the cornea 20, a change in incision of the anterior capsule 25 in the lens 23, a change when an interior (core or cortex) of the lens 23 is absorbed and removed, and the like. It should be noted that the change in condition of the eye can be any change as long as it is a change related to the condition of the eye irrespective of whether it is before/after surgical operation or during surgical operation.

In the example here, a correlation between the change in condition of the eye and the user's demand on the tomographic image is considered, and the parameters of the tomographic image can be changed in accordance with the change in condition of the eye so as to meet a user's demand. Specifically, the image recognition processing detects the change in condition of the eye shown in the front image and determines a speed of this change.

Then, if the speed of the change in condition of the eye is equal to or higher than the predetermined threshold, the frame rate priority mode is set. If the speed of the change in condition of the eye is slower than the predetermined threshold, the image quality priority mode is set. Also in the example here, actions and effects similar to the actions and effects in the first embodiment are exerted.

It should be noted that both of the motion (speed) of the surgical tool 7 and the change (speed) of the condition of the eye may be used and the parameter for acquiring the tomographic image may be changed.

### Second Embodiment

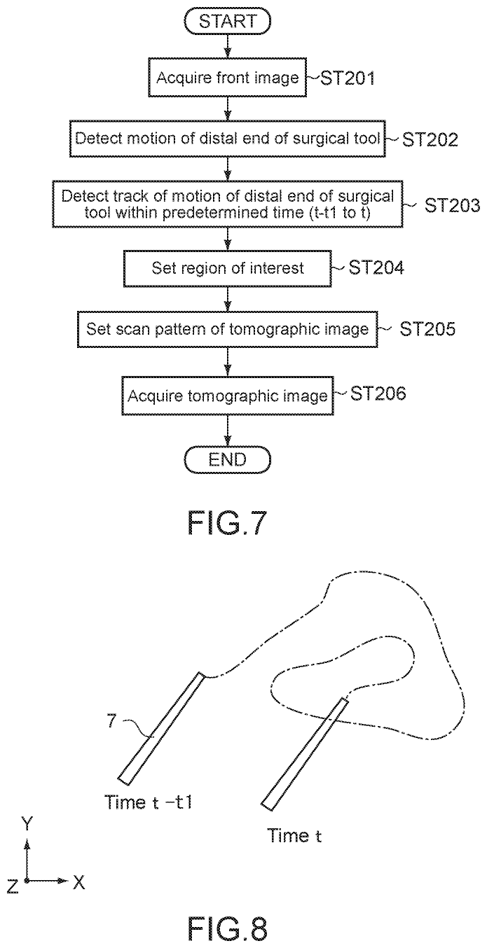

Next, a second embodiment of the present technology will be described. FIG. 7 is a flowchart showing processing according to the second embodiment.

As shown in FIG. 7, first of all, the control unit 1 controls the front image acquisition unit 2 to capture a front image of the eye and acquires the captured front image of the eye from the front image acquisition unit 2 (Step 201).

It should be noted that every time the front image is acquired, the control unit 1 causes the storage unit 4 to store the acquired front image. Further, the control unit 1 causes the acquired front image to be displayed on the screen of the display unit 5.

Next, the control unit 1 executes image recognition processing on the previous front image stored in the storage unit 4 and the acquired current front image (as in the first embodiment) and detects a motion of the distal end of the surgical tool 7 shown in the front image (Step 102).

When the control unit 1 detects the motion of the distal end of the surgical tool 7, the control unit 1 detects a track of the motion of the distal end of the surgical tool 7 between a time earlier than a current time t by a predetermined time t1 (e.g., approximately several seconds to several tens of seconds) and the current time t on the basis of the motion of the distal end of the surgical tool 7 (Step 203).

FIG. 8 is a diagram showing an example of the track of the motion of the distal end of the surgical tool 7 between the time earlier than the current time t by the predetermined time t1 and the current time t. In Step 203, the control unit 1 detects a track of the motion of the distal end of the surgical tool 7 as shown in FIG. 8.

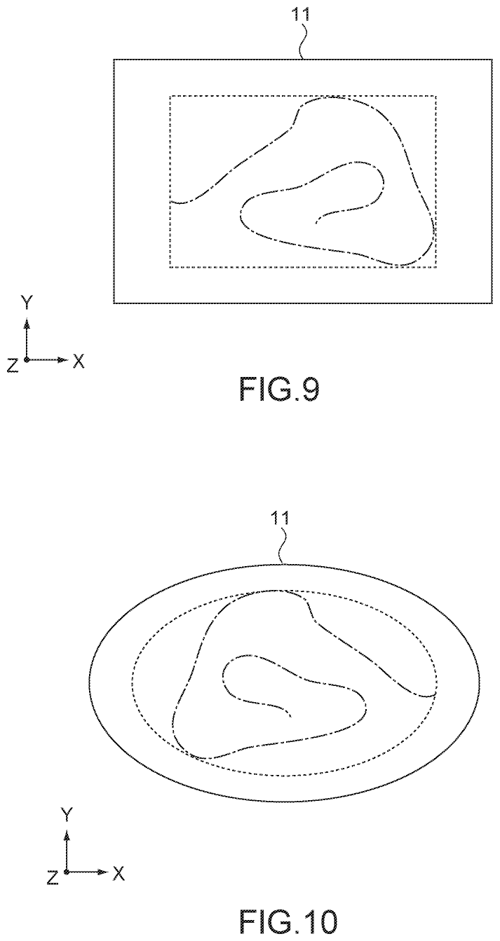

When the control unit 1 detects the motion of the distal end of the surgical tool 7, the control unit 1 sets a region of interest 11 (first region) on the basis of the track of the motion of the distal end of the surgical tool 7 (Step 204). FIGS. 9 and 10 are diagrams each showing when the region of interest 11 is set on the basis of the motion of the distal end of the surgical tool 7. In FIG. 9, a state when setting a rectangular region of interest 11 is shown. In FIG. 10, a state when an elliptical region of interest 11 is set is shown.

In setting the region of interest 11, first of all, the control unit 1 sets a region (see a rectangular shape or elliptical shape indicated as the broken line) surrounding the track by a rectangular shape, elliptical shape, or the like. Then, with respect to the region surrounding the track, the control unit 1 sets the region of interest 11 (see the rectangular shape or elliptical shape indicated as the broken line) by setting a marginal region outside this region. A fixed value may be used for the size of the marginal region. Alternatively, the size of the marginal region may be determined in a manner that depends on a ratio of the region surrounding the track to the size.

It should be noted that the shape of the region of interest 11 is not limited to the rectangular or elliptical shape, a polygonal shape or the like other than the rectangular shape may be used, and this shape is not particularly limited.

Here, the region of interest 11 is set to be within the measurement region 13. As described above, the measurement region 13 is a region in the plane direction in which the measurement by the OCT is performed (the region in the plane direction in which scan is performed).

FIG. 11 is a diagram showing a state when the region of interest 11 is set to be within the measurement region 13. In FIG. 11, the measurement region 13 is shown as a rectangular shape indicated as the long dashed short dashed line and the region of interest 11 is shown as a rectangular shape indicated as the solid line.

As shown in FIG. 11, in the second embodiment, scan is executed within the measurement region 13 including a predetermined region in the plane direction (the XY direction) for acquiring the tomographic image. That is, in this embodiment, scan is executed in two directions of the X-axis and Y-axis directions in the plane direction and the volume data (three-dimensional data) of the eye is acquired.

It should be noted that a polygonal shape or the like other than the elliptical shape and the rectangular shape may be used as the shape of the measurement region 13 and this shape is not particularly limited. Here, in the description below, the region other than the region of interest 11 will be referred to as a region of non-interest 12 (second region) within the measurement region 13.

When the region of interest 11 is set, then the control unit 1 generates a can pattern for acquiring the tomographic image on the basis of the set region of interest 11 (Step 205).

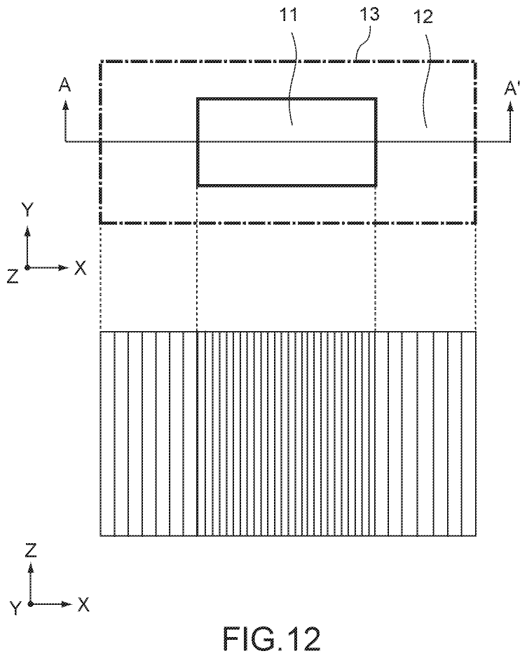

FIG. 12 is a diagram showing a scan pattern for acquiring the tomographic image. On the upper side of FIG. 12, a state when the measurement region 13 and the region of interest 11 are viewed from above is shown. On the other hand, on the lower side of FIG. 12, a state when the scan pattern in acquiring the tomographic image is viewed from the side (a cross-section taken along A-A′ in the upper representation of FIG. 12) is shown.

As shown in FIG. 12, in the measurement region 13, the scan density in the plane direction (the XY direction) is different between the region of interest 11 and the region of non-interest 12. That is, the control unit 1 changes the scan density (parameter) between the region of interest 11 and the region of non-interest 12. Specifically, the control unit 1 changes the scan density in the scan such that the scan density in the region of interest 11 becomes higher than the scan density in the region of non-interest 12.

When the scan pattern is set, then the control unit 1 causes the tomographic image acquisition unit 3 to acquire the tomographic image in accordance with the set scan pattern (Step 206). It should be noted that the region of interest 11 has scan density higher than that of the region of non-interest 12. Therefore, in the tomographic image, the image quality of the region of interest 11 is higher than the image quality of the region of non-interest 12.

Here, the region (region of interest 11) included in the track of the motion in the distal end of the surgical tool 7 can be considered as being a region for which the demand of specifically observing the condition of the eye from the high-quality tomographic image from the perspective of the user is present. That is, also in the second embodiment, as in the first embodiment, the correlation between the motion of the surgical tool 7 and the user's demand is considered and the parameter (scan density) of the tomographic image is suitably changed between the region of interest 11 and the region of non-interest 12 in accordance with the motion of the surgical tool 7 so as to meet a user's demand.

Therefore, also in the second embodiment, as in the first embodiment, it is possible to set a suitable parameter for acquiring a tomographic image that meets a user's demand in accordance with the motion of the surgical tool 7. In particular, in the second embodiment, in the tomographic image, the user can specifically observe the condition of the eye in the region of interest 11 in which the surgical operation is being performed while observing the entire eye in the measurement region 13 without requiring complicated work.

### Modified Example of Second Embodiment

“Example of Making Frame Rate Different between Region of Interest 11 And Region of Non-Interest 12”

In the above description, the case where the image quality of the region of interest 11 and the image quality of the region of non-interest 12 are made different has been described. On the other hand, the frame rate of the region of interest 11 and the frame rate of the region of non-interest 12 may be different.

That is, the control unit 1 may change the frame rate (parameter) in the tomographic image between the region of interest 11 and the region of non-interest 12. In this case, the control unit 1 may change the frame rate such that the frame rate in the region of interest 11 is higher than the frame rate in the region of non-interest 12.

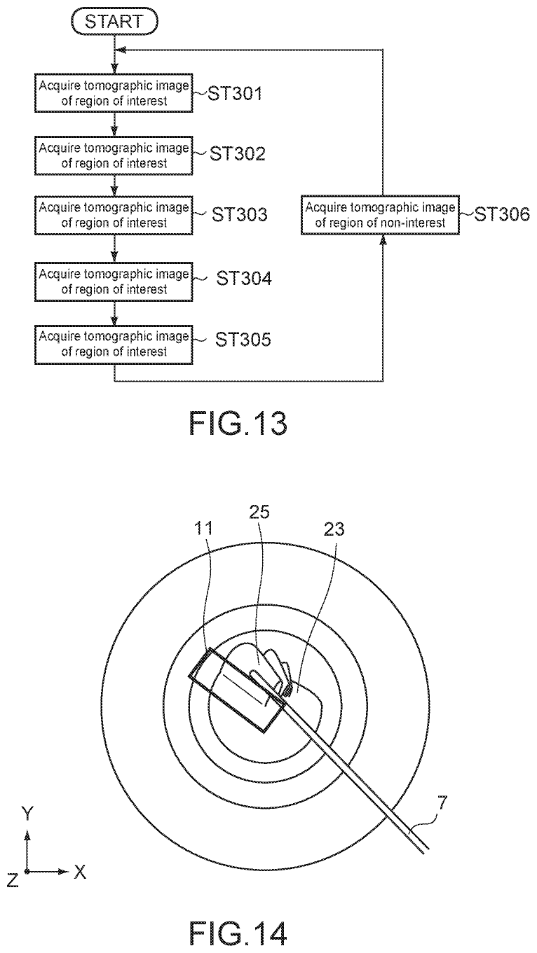

Hereinafter, it will be described specifically by showing an example. FIG. 13 is a flowchart showing processing in a case where the frame rate of the region of interest 11 and the frame rate of the region of non-interest 12 are made different.

After the control unit 1 sets the region of interest 11 within the measurement region 13, the control unit 1 repeats processing of acquiring the tomographic image corresponding to the region of interest 11 through the tomographic image acquisition unit 3 five times as shown in FIG. 13 (Steps 301 to 305). Every time the control unit 1 acquires the tomographic image corresponding to the region of interest 11, the control unit 1 updates the tomographic image regarding a position corresponding to the region of interest 11 in the tomographic image.

After the control unit 1 repeats the above-mentioned processing five times, the control unit 1 acquires the tomographic image corresponding to the region of non-interest 12 through the tomographic image acquisition unit 3 (Step 306).

By such processing, the tomographic image corresponding to the region of interest 11 is updated five times while the tomographic image corresponding to the region of non-interest 12 is updated once. That is, the frame rate in the region of interest 11 is five times as high as the frame rate in the region of non-interest 12.

The magnification of the frame rate of the region of interest 11 to the frame rate of the region of non-interest 12 is not limited to the five times and can be changed as appropriate. It should be noted that although in the example here, the scan density of the region of interest 11 and the scan density of the region of non-interest 12 are the same, the scan density may be different.

In this example, the user can quickly grasp the change in condition of the eye in the region of interest 11 in which the surgical operation is being performed while observing the entire eye in the measurement region 13 in the tomographic image.

[Example of Setting Region of Interest 11 on Basis of Variation of Condition of Eye]

In the description above, the case where the region of interest 11 is set on the basis of the motion of the surgical tool 7 has been described. On the other hand, the region of interest 11 may be set on the basis of the change in condition of the eye.

That is, a region of the front image, in which the condition of the eye is changing, can be considered as being a region for which the demand of quickly grasping a change in condition of the eye or the demand of specifically observing the condition of the eye is present. Therefore, this relationship may be utilized and the region of interest 11 may be set on the basis of the change in condition of the eye.

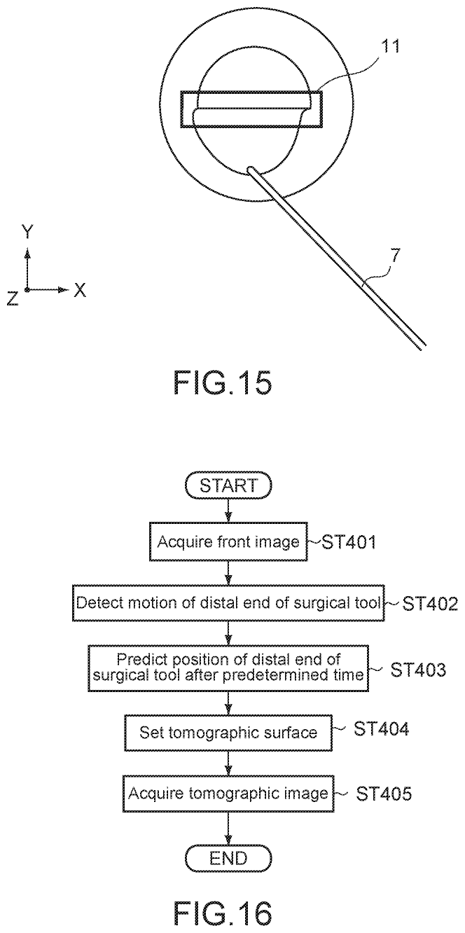

FIGS. 14 and 15 are diagrams each showing when the region of interest 11 is set on the basis of the change in condition of the eye. In FIG. 14, a change in condition of the eye when incision of the anterior capsule 25 in the lens 23 is being performed in cataract surgery is shown. In FIG. 15, a change in condition of the eye when an inner limiting membrane in the vitreous body is peeled in vitreoretinal surgery is shown.

In this case, the control unit 1 executes image recognition processing on the previous front image stored in the storage unit 4 and the acquired current front image and detects a change in condition of the eye shown in the front image. Then, the control unit 1 sets the region of interest 11 so as to surround a portion in which the condition of the eye is changing. In setting the region of interest 11, a marginal region may be set.

For example, in the example shown in FIG. 14, incision of the anterior capsule 25 in the lens 23 is being performed through the surgical tool 7. The portion in which the membrane of the anterior capsule 25 is being peeled is determined as the portion in which the condition of the eye is changing and the region of interest 11 is set so as to surround this portion.

Further, in the example shown in FIG. 15, the inner limiting membrane in the vitreous body is being peeled through the surgical tool 7. The portion in which the inner limiting membrane is being peeled is determined as the portion in which the condition of the eye is changing and the region of interest 11 is set so as to surround this portion.

In the examples shown in FIGS. 14 and 15, the region of interest 11 is set to have a rectangular shape. The shape of the region of interest 11 is not particularly limited.

When the region of interest 11 is set, the control unit 1 makes the image quality or the frame rate (or both of them) different between the region of interest 11 and the region of non-interest 12.

In the example here, the user can easily check a drawing state of the membrane, a state of bonding between the membrane and other tissues, and the like in the tomographic image. Therefore, it is possible to easily recognize whether or not unnecessary force is added, for example.

### Third Embodiment

Next, a third embodiment of the present technology will be described. In the third embodiment, the motion of the surgical tool 7 is predicted and the tomographic surface (parameter) for acquiring the tomographic image is changed in accordance with a prediction result.

FIG. 16 is a flowchart showing processing according to a third embodiment. As shown in FIG. 16, first of all, the control unit 1 controls the front image acquisition unit 2 to capture a front image of the eye and acquires the captured front image of the eye from the front image acquisition unit 2 (Step 401).

It should be noted that every time the front image is acquired, the control unit 1 causes the storage unit 4 to store the acquired front image. Further, the control unit 1 causes the acquired front image to be displayed on the screen of the display unit 5.

Next, the control unit 1 executes image recognition processing (as in the first embodiment) in the previous front image stored in the storage unit 4 and the acquired current front image and detects a motion of the distal end of the surgical tool 7 shown in the front image (Step 402).

When the control unit 1 detects the distal end of the motion of the surgical tool 7, the control unit 1 predicts a position of the distal end of the surgical tool 7 at a time (t+dt) later than the current time by a predetermined time on the basis of the motion of the distal end of the surgical tool 7 (Step 403).

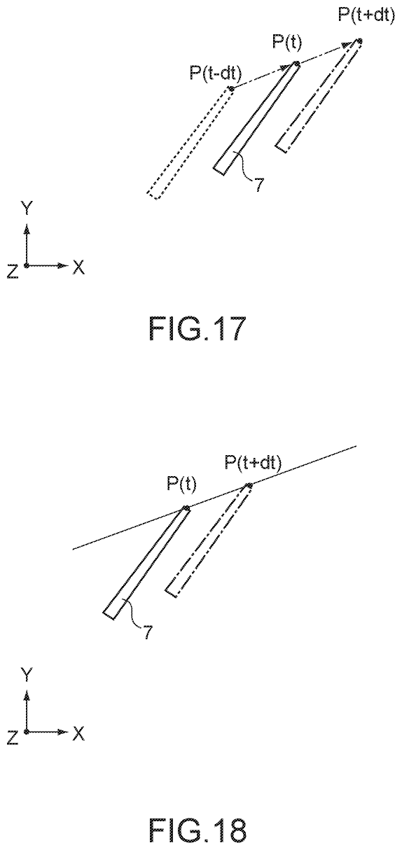

FIG. 17 is a diagram showing a state when the position of the distal end of the surgical tool 7 at the time later than the current time by the predetermined time is predicted. In FIG. 17, the surgical tool 7 at the current time t is indicated as the solid line, the surgical tool 7 at a previous time (t−dt) is indicated as the broken line, and the surgical tool 7 at the time (t+dt) later than the current time by the predetermined time is indicated as the long dashed short dashed line.

Further, a position of the distal end of the surgical tool 7 at the current time t is indicated by P(t). A position of the distal end of the surgical tool 7 at the previous time (t−dt) is indicated by P(t−dt). A predicted position of the distal end of the surgical tool 7 at the time (t+dt) later than the current time by the predetermined time is indicated by P(t+dt). It should be noted that dt is typically set to be the same time as the frame rate.

In the example shown in FIG. 17, the control unit 1 detects a motion (track) of the distal end of the surgical tool 7 on the basis of the position P(t) of the distal end of the surgical tool 7 at the current time and the position P(t−dt) of the distal end of the surgical tool 7 at the previous time (and the position of the distal end at a time earlier than the previous time) (see Step 402). Then, on the basis of this motion, the control unit 1 predicts a position P(t+dt) of the distal end of the surgical tool 7 at the time (t+dt) later than the current time by the predetermined time (see Step 403).

When the control unit 1 predicts a position P(t+dt) of the distal end of the surgical tool 7 at the time (t+dt) later than the current time by the predetermined time, the control unit 1 sets a tomographic surface for acquiring the tomographic image on the basis of the prediction result.

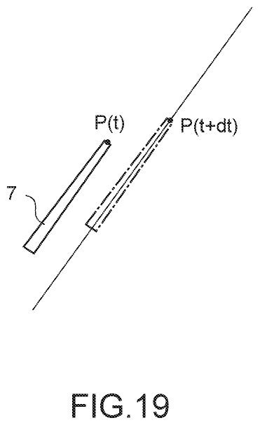

FIGS. 18 and 19 are diagrams each showing an example of the tomographic surface determined on the basis of the prediction result. In FIG. 18, an example of a case where a straight line linking the position P(t) of the distal end of the surgical tool 7 at the current time and the predicted position P(t+dt) of the distal end of the surgical tool 7 at the time (t+dt) later than the current time by the predetermined time is set as the tomographic surface is shown.

On the other hand, in FIG. 19, an example of a case where the straight line including the predicted position P(t+dt) of the distal end of the surgical tool 7 at the time (t+dt) later than the current time by the predetermined time is set as the tomographic surface is shown. It should be noted that although in FIG. 19, the tomographic surface is set to be along the longitudinal direction of the surgical tool 7, the tomographic surface may be any straight line as long as it is a straight line including the predicted position P(t+dt) of the distal end of the surgical tool 7.

When the tomographic surface is set, the control unit 1 causes the tomographic image acquisition unit 3 to acquire the tomographic image according to the set tomographic surface (Step 405).

In the third embodiment, the tomographic surface can be set at a suitable position. Also when the motion of the surgical tool 7 is speedy, the distal end of the surgical tool 7 can be included in the tomographic image.

### VARIOUS MODIFIED EXAMPLES

In the description above, the case where the control unit 1 in the surgical microscope apparatus 10 executes the above-mentioned various types of processing has been described. On the other hand, the above-mentioned various types of processing may be executed by the control unit 1 of the server apparatus over the network (information processing apparatus).

The present technology can also take the following configurations.

(1) An information processing apparatus, including

a control unit that detects a time-series change of an object shown in an image obtained by taking an image of an eye which is a surgical operation target and changes a parameter for acquiring a tomographic image of the eye in accordance with the time-series change of the object.

(2) The information processing apparatus according to (1), in which

the control unit detects a change in position of a surgical tool as the time-series change of the object and changes the parameter in accordance with the change in position of the surgical tool.

(3) The information processing apparatus according to (1) or (2), in which

the control unit detects a change in condition of the eye as the time-series change of the object and changes the parameter in accordance with the change in condition of the eye.

(4) The information processing apparatus according to any one of (1) to (3), in which

the control unit switches between a first mode on which the parameter is set such that priority is given to a frame rate over an image quality in the tomographic image and a second mode on which the parameter is set such that priority is given to the image quality over the frame rate in the tomographic image in accordance with the time-series change of the object.

(5) The information processing apparatus according to (4), in which

the control unit detects a change speed in the time-series change of the object and switches between the first mode and the second mode in accordance with the change speed.

(6) The information processing apparatus according to (5), in which

the control unit determines whether or not the change speed is equal to or higher than a predetermined threshold, sets the first mode if the change speed is equal to or higher than the predetermined threshold, and sets the second mode if the change speed is lower than the predetermined threshold.

(7) The information processing apparatus according to any one of (1) and (6), in which

the control unit sets, in accordance with the time-series change of the object, a first region within a measurement region in which scan for obtaining the tomographic image is performed and changes the parameter between the first region and a second region, the second region being a region other than the first region within the measurement region.

(8) The information processing apparatus according to (7), in which

the control unit changes scan density in the scan as the parameter between the first region and the second region.

(9) The information processing apparatus according to (8), in which

the control unit changes the scan density in the scan such that scan density of the first region is higher than scan density of the second region.

(10) The information processing apparatus according to (7), in which

the control unit changes a frame rate in the tomographic image as the parameter between the first region and the second region.

(11) The information processing apparatus according to (9), in which

the control unit changes a frame rate in the tomographic image such that a frame rate in the first region is higher than a frame rate in the second region.

(12) The information processing apparatus according to any one of (1) to (11), in which

the control unit changes the tomographic surface for acquiring the tomographic image as the parameter on the basis of the time-series change of the object.

(13) The information processing apparatus according to (12), in which

the control unit predicts a change of the object at a time later than a current time by a predetermined time in accordance with the time-series change of the object and changes the tomographic surface in accordance with a prediction result.

(14) An information processing method, including:

detecting a time-series change of an object shown in an image obtained by taking an image of an eye which is a surgical operation target; and

changing a parameter for acquiring a tomographic image of the eye in accordance with the time-series change of the object.

(15) A program that causes a computer to execute:

a step of detecting a time-series change of an object shown in an image obtained by taking an image of an eye which is a surgical operation target; and

a step of changing a parameter for acquiring a tomographic image of the eye in accordance with the time-series change of the object.

### REFERENCE SIGNS LIST

1 control unit
2 front image acquisition unit
3 tomographic image acquisition unit
7 surgical tool
10 surgical microscope apparatus
11 region of interest
12 region of non-interest

1. An information processing apparatus, comprising
a control unit that detects a time-series change of an object shown in an image obtained by taking an image of an eye which is a surgical operation target and changes a parameter for acquiring a tomographic image of the eye in accordance with the time-series change of the object.

2. The information processing apparatus according to claim 1, wherein
the control unit detects a change in position of a surgical tool as the time-series change of the object and changes the parameter in accordance with the change in position of the surgical tool.

3. The information processing apparatus according to claim 1, wherein
the control unit detects a change in condition of the eye as the time-series change of the object and changes the parameter in accordance with the change in condition of the eye.

4. The information processing apparatus according to claim 1, wherein
the control unit switches between a first mode on which the parameter is set such that priority is given to a frame rate over an image quality in the tomographic image and a second mode on which the parameter is set such that priority is given to the image quality over the frame rate in the tomographic image in accordance with the time-series change of the object.

5. The information processing apparatus according to claim 4, wherein
the control unit detects a change speed in the time-series change of the object and switches between the first mode and the second mode in accordance with the change speed.

6. The information processing apparatus according to claim 5, wherein
the control unit determines whether or not the change speed is equal to or higher than a predetermined threshold, sets the first mode if the change speed is equal to or higher than the predetermined threshold, and sets the second mode if the change speed is lower than the predetermined threshold.

7. The information processing apparatus according to claim 1, wherein
the control unit sets, in accordance with the time-series change of the object, a first region within a measurement region in which scan for obtaining the tomographic image is performed and changes the parameter between the first region and a second region, the second region being a region other than the first region within the measurement region.

8. The information processing apparatus according to claim 7, wherein
the control unit changes scan density in the scan as the parameter between the first region and the second region.

9. The information processing apparatus according to claim 8, wherein
the control unit changes the scan density in the scan such that scan density of the first region is higher than scan density of the second region.

10. The information processing apparatus according to claim 7, wherein
the control unit changes a frame rate in the tomographic image as the parameter between the first region and the second region.

11. The information processing apparatus according to claim 9, wherein
the control unit changes a frame rate in the tomographic image such that a frame rate in the first region is higher than a frame rate in the second region.

12. The information processing apparatus according to claim 1, wherein
the control unit changes the tomographic surface for acquiring the tomographic image as the parameter on a basis of the time-series change of the object.

13. The information processing apparatus according to claim 12, wherein
the control unit predicts a change of the object at a time later than a current time by a predetermined time in accordance with the time-series change of the object and changes the tomographic surface in accordance with a prediction result.

14. An information processing method, comprising:
detecting a time-series change of an object shown in an image obtained by taking an image of an eye which is a surgical operation target; and
changing a parameter for acquiring a tomographic image of the eye in accordance with the time-series change of the object.

15. A program that causes a computer to execute:
a step of detecting a time-series change of an object shown in an image obtained by taking an image of an eye which is a surgical operation target; and
a step of changing a parameter for acquiring a tomographic image of the eye in accordance with the time-series change of the object.
